(19) United States
(12) Patent Application Publication
(10) Pub. No.: US 2017/0231675 A1
(43) Pub. Date: Aug. 17, 2017

(54) BONE SCREW

(71) Applicant: Z-MEDICAL GMBH & CO. KG, Tuttlingen (DE)

(72) Inventor: Zbigniew Combrowski, Tuttlingen (DE)

(21) Appl. No.: 15/477,673

(22) Filed: Apr. 3, 2017

Related U.S. Application Data
(62) Division of application No. 13/376,815, filed on Mar. 1, 2012.

(30) Foreign Application Priority Data
Jun. 8, 2009 (DE) 102009024554.5
May 5, 2010 (DE) 102010016812.2

(51) Int. Cl. A61B 17/86; A61B 17/88

(52) U.S. Cl. CPC A61B 17/8615; A61B 17/864; A61B 17/8888; A61B 17/888; A61B 17/8635; A61B 17/863

(57) ABSTRACT

The invention relates to a surgical instrument comprising a bone screw having a screw head, and a shaft for rotating the bone screw in a bone, the shaft and/or head forming a single element with the bone screw and having a predetermined breaking point. A driving element is inserted into the bone screw by the shaft, said driving element screwing the screw further in or out once the shaft has broken off from the screw.

### CROSS-REFERENCE TO RELATED APPLICATION

This application is a divisional application, and claims priority to U.S. application Ser. No. 13/376,815, filed Mar. 1, 2012, which is a national stage filing based upon International PCT Application No. PCT/EP2010/003421, with an international filing date of Jun. 8, 2010, which claims the benefit of priority to German Application No. 102010016812.2, filed May 5, 2010 and German Application No. 102009024554.5, filed Jun. 8, 2009, all of which are fully incorporated herein by reference as though fully set forth herein.

### TECHNICAL FIELD

The present inventions relates to a surgical instrument.

### BACKGROUND

In the prior art, it is known to connect bone screws integrally with an auxiliary material for positioning the bone screw in a bone. With the exceedance of a specific torque the auxiliary material shears off at a predetermined breakage point from the bone screw.

For instance, the patent document U.S. Pat. No. 6,808,526 reveals a bone screw for fixing of bones, with a grip similar to a screw driver, which is connected through a predetermined breakage point. With the exceedance of a specific torque the grip/knob shears at the predetermined breaking point. Nevertheless this grip/knob is very thin, short and rigidly executed. The disadvantage is that the force for shear-off is relatively small and/or is not exactly defined as it detaches during the breakage.

Besides the shear-off point is increased and presents a defect, which can lead to irritation in tissue. With this bone screw there is also the danger that it shears-off prematurely with an unevenly applied force or with an angle error while inserting it. Further insertion is possible only with great difficulty. Eventually, the screw must be actually removed associated with additional expenses and with disadvantage for the patient.

### SUMMARY

The objective of the present invention is to create a surgical instrument or positioning tool that can be positioned in a simple manner and with which the shear force is possibly defined, lesser force is required for inserting a screw, positioning error with screw-in does not immediately lead to a premature breakage and the shearing point restricts the healing process to the minimum extent possible. Besides, no additional expenditure occurs with sterilization and still higher requirements for sterility are taken into account. Furthermore, the process safety of OP is improved to that effect that the possibility of keeping the bone screw in the body till the onset/attachment in the bones is avoided.

For solving the present objective, the characterizing part of the Claim 1 leads.

In a preferred embodiment, a surgical instrument as per the invention presents a bone screw with a screw head and a shaft for screwing-in the bone screw in a bone.

In a preferred embodiment, a surgical instrument as per the invention presents a shaft which is shaped as a grip/knob. The advantage here is that the handling of the surgical instrument is simplified.

The screw implant as per the invention with a shaft and/or handle/grip/knob in a preferred embodiment presents a multi-part handle/grip/knob, in which the handle is extractable preferably in longitudinal direction, so that the operator can screw-in at any point of time every screw implant individually in the desired position without additional instruments.

In an embodiment, the bone screw can present a single or multiple threading. This offers the possibility of a wide range of applicability of such a bone screw.

In another embodiment, the invention-based bone screw can be implemented as cannulated, in doing so this cannulation comprises cross-holes from a channel running from the inner side of the bone screw. This cannulation, together with the cross-hole offers the advantage that medically active substances can be directly administered into the bones through the shaft and subsequently through the screw head and the cannulation existing there and the cross-holes. Besides, in the same way a filler material is administered for supporting the bone and for better joining in the bones. As filler material, a fluid cement or plaster specially created for this purpose is considered. These screws are usable both for open Ops and also for minimum invasive interventions.

In a preferred embodiment, the shaft and the bone screw are developed as a single unit. This has the advantage that a sterile surgical instrument is delivered directly to the user, who can dispose-off the shaft after implanting the screw.

Another preferred embodiment presents a predetermined breaking point between the shaft and the screw head of the bone screw. This predetermined breaking point is capable of breaking with the bending of the shaft or after reaching a definite torque. The required power/torque for bending the shaft is to be adjusted to the requirements at the bone screw in the hospital. A resetting of the screw after the breakage of the shaft and/or handle is possible at any point of time outside or inside the screw head or inside the cannulation.

In another preferred embodiment of a surgical instrument, the predetermined breaking point is arranged in the form of a ring-shaped sunk-in groove/predetermined breaking point of the screw head. The depth of the groove/predetermined breaking point is preferably between 0.01 mm and 10 mm, yet a depth between 0.1 mm and 10 mm is most preferred. The depth of the groove/predetermined breaking point is to be ascertained from the highest point of the screw head. Advantageously, the fact is that the required power for bending/twisting the shaft from the screw head is reduced at the groove/predetermined breaking point and in the reverse the predetermined breaking point can be executed in a stable manner that the user while inserting the bone screw can still apply sufficient force in order to rotate the screw in the bone mass. It is also conceivable, to design the ring-shaped predetermined breaking point stably for higher torque and to open or shear-off the predetermined breaking point with the help of a special tong.

The surgical instrument as per the invention presents as auxiliary material for insertion, a shaft, and a bone screw that later remains in the bone, with an advantageously self-cutting threading. In the process the shaft and the bone screw area-wise present a contour, which is designed in such a way that it can be held with invention-based surgical instrument or can be brought into operative connection. That means, after shearing-off the shaft from the screw part remaining in the bone, the shaft as per the invention can be advantageously positioned without change in its positioning or other adjustment measures at the bone screw and insert the bone screw further in to the bone.

Likewise, the surgical instrument as per the invention serves in reimplantation of the bone screw. For this purpose, a suitable contour is applied on the screw part and rotated opposite to the direction of rotation.

A preferred embodiment presents an adapter in the screw head. This adapter can present different forms. A specially preferred adapter represents itself as inner square or a hexagonal drive. In this connection, it is advantageous that the user can initially screw-in the bone screw into the bone through a counter piece/multi-point screw driver in the form of shaft corresponding to the inner serration and the shaft is subsequently removed. It is also advantageous that, the user can screw-in the screw into the bone screw into the bone or take out the bone from the screw by subsequent resetting of shaft.

Another preferred embodiment presents a shaft, which presents a hole in longitudinal direction, through which an inner lying adapter with inner cross serration can be inserted in the screw head. The advantage is that a maximum possible loss-free and friction-free power transfer can be achieved.

With another preferred embodiment, the adapter in the screw head presents a recess or an additional groove. This recess or additional groove offers the advantage that the adapter can be prepared without residual burr, shavings and thus no dead space results that can no longer be sterilized.

Another preferred embodiment presents a connector, which is developed as part of the shaft. This connector is to be suitable to co-act with a knob or a rod. This can be executed in the form of a plug system. In this connection, it is important that the knob is connected with the shaft in such a way that the user can actuate the shaft and thereby the bone screw. Subsequently the user should be able to remove the knob individually or can be bent together with the shaft from the bone screw.

Another embodiment of the invention-based knob is developed as multi-part knob. This can mean that the knob comprises of different separable parts. It can also mean that the knob consists of parts countersunk parts in each other which can be extended as a telescope, in doing so the complete length of the knob enables to be changed. Advantageous in this case is a possible multi-purpose use of the knob.

In a preferred embodiment, the shaft and knob are designed as disposable articles and should be disposed off after screwing-in the bone screw. This gives the advantage of a sterile usage and subsequently simple disposal.

The bone screw and the shaft are delivered as single unit and sterile. The user can set up the knob and rotate the bone screw into the bone with the help of the shaft. The knob can also be designed as reusable article. In doing so, the advantage is that no additional sterilization of the corresponding devices of the prior art is required any longer.

The bone screw can be designed as cannulated or non-cannulated. Likewise, the shaft and/or the knob can be designed as cannulated or only as hollow. The difference is that, with a cannulation a channel from the top end of the shaft, the place which possesses the connector with the knob and the bottom end of the shaft, which connects to the bone screw. With a hollow shaft and/or knob the top or the bottom area are also designed as closed.

In a preferred embodiment the bone screw possesses a self-drilling tip and/or a self-snapping threading. Here, the advantage is that the bone screw can be simply screwed-in in the bones and the expenditure of effort of the user is reduced.

The shaft for implantation and screw part of the bone screw remaining in the bones are integrally connected through a predetermined breaking point. Preferably, a hole is made in the shaft so that the shaft and the screw head are connected only through a ring-like area and/or shear-off area.

With the term “annular”, in the present application not only the round forms are described but all forms which present a recess or a section in the inner area. Even square, oval or irregular frames fall under the description of “annular”. For instance, here a rectangle with a rectangle section or an oval with a round section are to be mentioned. From the concept of invention, it also includes an annular connecting area, i.e., the predetermined breaking point is designed as perforated.

The drilling in shaft enables to place an inner serration in screw head on the one hand, at the same time, through the annular design of the predetermined breaking point a defined lever/lever arm emerges over the radius, and thereby a defined moment results for breaking the shaft from the bone screw with high directional stability. Another advantage of the hole in the shaft is that a wrong positioning of the bone screw with the implantation leads to a premature shear-off with comparable screws as a higher lateral force is achievable over the radius of the hole.

The predetermined breaking point conveniently sunk in a screw head of the screw part remaining in the bone. This has the advantage that even the shearing point is later sunk in the screw head and cannot lead to irritation with its rough surface in the surrounding tissue, like bones, skin or flesh.

The threading of the bone screw is preferably designed conically. As a result, the bone screw can be easily implanted and re-implanted. It will require lesser force for rotating and/or cutting the threading with implantation. Another advantage of a conical threading is that it results in better compression. Likewise, multiple threading are included in the concept of the invention.

The screw driver as per the invention, for which claim is separately made, preferably presents an attachment/holder/clamp, preferably co-acts with a recess but also an elevation is to be included by the inventory concept. The holder is designed advantageously in such a way that it also serves as an ejector for the sheared shaft.

The bone screw and the screw driver as per the invention form an assembly set, for which separate claim is made. Advantageously, this assembly set comprises a magazine/set for protection of bone screws against rotation. The advantage of such a magazine is that the sterilized screws must be handled without touching by hand, they can be taken out only with screw driver and implanted directly.

A preferred embodiment for the rod is a cylindrical part, which is shaped either as hollow pipe or as fully synthetic material or as cylindrical part with a cannulation. The advantage is that the rod simplifies its work of the user through its longitudinal shape as the bone screw can be screwed-in or taken out without forcing the user to intervene in the tissues surrounding the bones.

### BRIEF DESCRIPTION OF DRAWINGS

Further advantages, features and details of the invention result from the following description of the embodiments as well as with the aid of the drawing; this shows in FIG. 1 a top view of a bone screw as per the invention;

FIG. 2 a section along a line II-II in FIG. 1;

FIG. 3 a perspective view of the invention-based bone screw as per FIG. 1;

FIG. 4 a lateral view of a screw driver as per the invention with the bone screw as per the invention;

FIG. 5 a top view of the invention-based screw driver as per FIG. 4 rotated around 90°;

FIG. 6 a perspective illustration of a position for a screw driver as per the invention;

FIG. 7 a perspective view of a screw driver as per the invention with positioning as per FIG. 6;

FIG. 8 an enlarged section from the screw driver as per FIG. 4 in the application field;

FIG. 9 a front view of a screw driver as per the invention with an invention-based bone screw and a magazine as per the invention.

FIG. 10 a cutaway lateral view of a surgical instrument as per the invention and a knob;

FIG. 11 an enlarged lateral view of a section from FIG. 10 from top view;

FIG. 12 a cut lateral view of another embodiment of a separate surgical instrument;

FIG. 13 an enlarged view inclined above a part of an assembled surgical instrument as per FIG. 12;

FIG. 14 a sectional lateral view of a surgical instrument as per FIG. 10 with set knob;

FIG. 15 an enlarged lateral view of a part of surgical instrument as per FIG. 14.

### DETAILED DESCRIPTION

FIG. 1 shows a bone screw 1 as per the invention. This presents a shaft 2 as auxiliary material for positioning and a screw part 3. The screw part 3 comprises of a screw head 5 and a screw shaft 7. The screw shaft 7 of the screw part 3 is provided over the ca.⅔of its length with a self-cutting threading 6.

The shaft 2 is integrally connected over a predetermined breaking point 4 at the screw head 5 with the screw part 3. At the periphery of a sleeve section 30, the shaft 2 presents a surrounding groove 8 and a projection 9 shaped at the sleeve section 30. The projection 9 tapers at an end 28 at a diameter D, smaller than a diameter d of the shaft 2 and is connected with this tapered end 28 integrally with the screw part 3 and to the screw head 5. The tapered end 28 lies somewhat deep in a depression 29 in the screw head 5 and represents the predetermined breaking point 4.

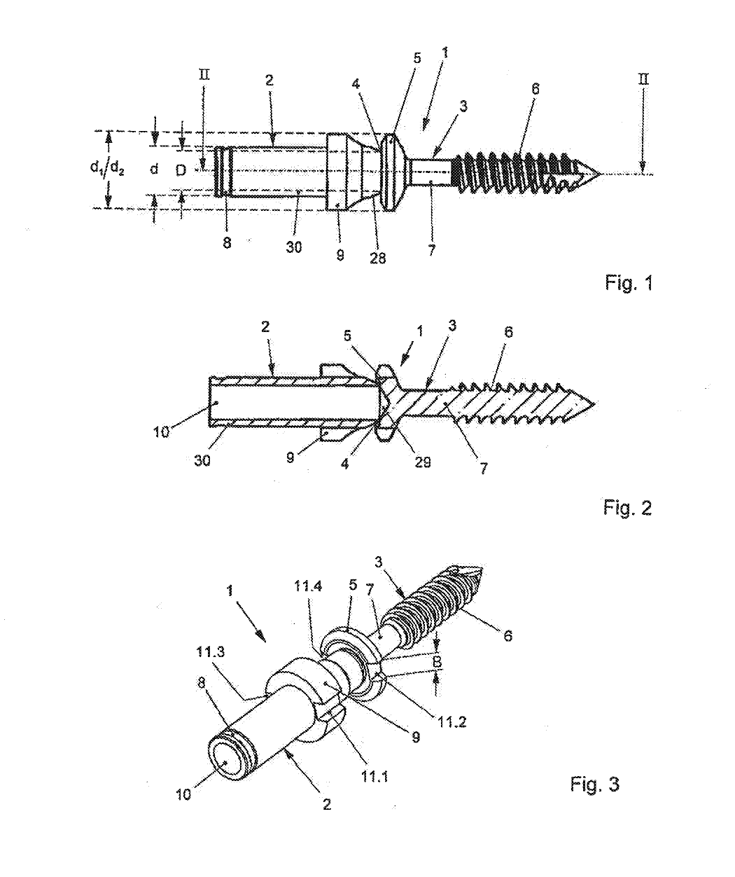

As the section in FIG. 2 shows, the shaft 2 presents a central bore 10. This bore 10 goes through the shaft 2 so that a shearing area is annularly formed at the predetermined breaking point 4.

The screw head 5 presents a similar external contour like the section 9. In the screw head 5 and in the section 9 two square notches 11.1 and 11.3 as well as 11.2 and 11.4 are made with a breadth B. As shown in FIG. 3, the notches 11.1 and 11.2 align the opposite lying notches 11.3 and 11.4

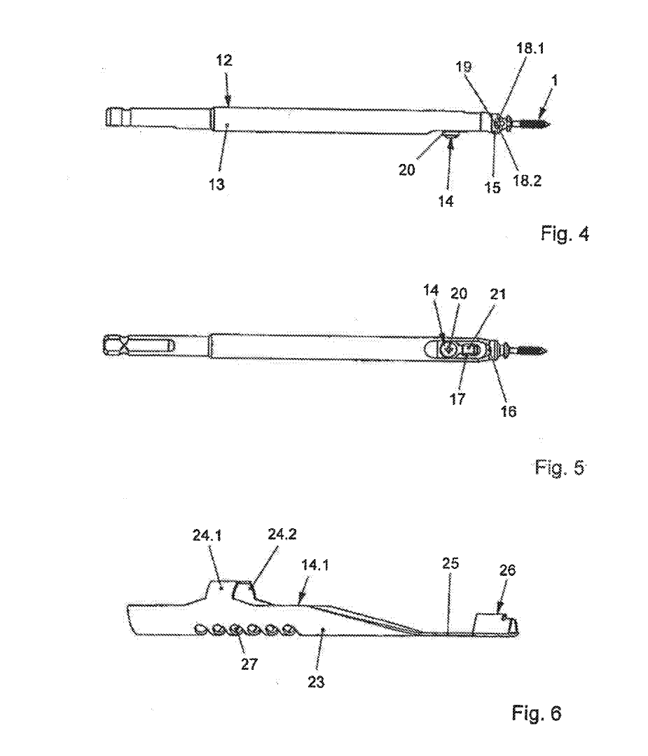

Further FIG. 4 shows an invention-based screw driver 12 for implantation of an invention-based bone screw 1, for which separate claim is made. The screw driver 12 presents a tubular base part 13, a holder 14 and a shaft and screw slot 15.

In its front area the shaft and the screw slot 15, two axial, square-shaped sections 18.1 and 18.2 are created in such a way that between them a lug 19 is developed. Not shown here, two such opposite lying sections and a corresponding lug are arranged. In doing so the lug 19 is narrower than the width B of the notches 11.1 To 11.4, so that the lugs 19 can engage into the opposite lying notches 11.1 and 11.3 in the shaft 2 or the notches 11.2 and 11.4 in the screw head 5 respectively.

For the holder 14, a recess 16 is created in the tubular base part 13, which is designed as solid material and in to the screw recess 15, which is designed as hollow material. In the hollow material of the screw and shaft recess 15, an aperture 17 is provided. In the solid material of the base part 13, a clamping screw 20 is fitted with the aid of a bracket 21. The bracket 21 projects into the recess 16 and through the aperture 17 into an inner space of the shaft and screw recess 15. In doing so, the bracket 21 is bent in such a way that it can engage into the groove 8 in shaft 2 of the bone screw 1. Thus, the bone screw 1 is held in the screw driver 12.

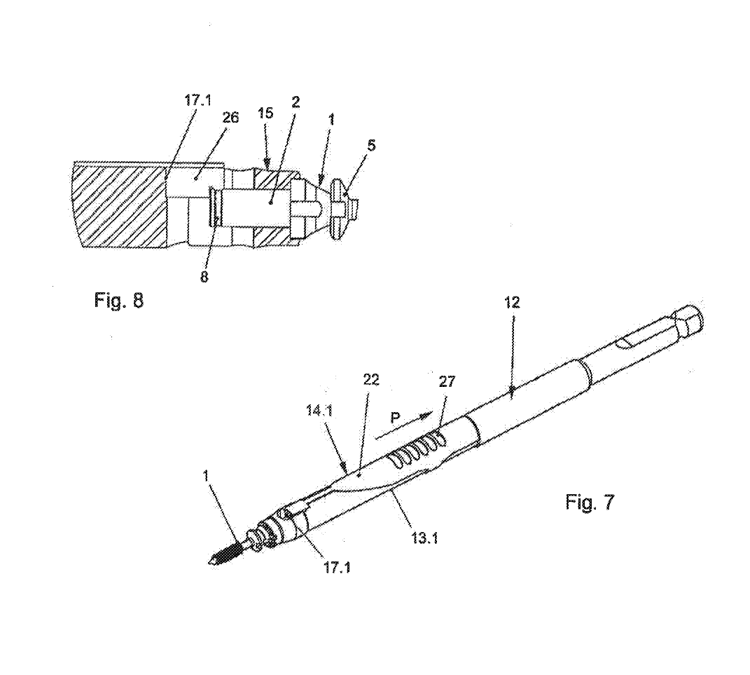

FIG. 7 shows another, preferred embodiment as per the invention of a screw driver 12.1. The screw driver 12.1 conforms essentially to the invention-based screw driver 12, but distinguishes itself in the design of a holder 14.1.

The holder 14.1 is an integrally-formed part from a spring (see FIG. 6), formed and presents a shell-like base 23 with retaining plate 24.1 and 24.2. In the base parts 23, slit 27 is introduced in order to ensure a better grip, to increase flexibility and to save weight and material.

The base part 23 ends in a narrow web 25, which forms a kind of hook 26 at its tip. The holder 14.1 is placed with the shell-like base part 23 and the retaining plates 24.1 and 24.2 around a tubular base part 13.1 of the screw driver 12.1 In the process the hook 26 grips, as clearly evident from the section in FIG. 8, through an aperture 17.1 and into the groove 8 in the bone screw 1.

The functionality of the present invention is given below:

For implantation of the bone screw 1 as per the invention, this is set in an invention-based screw driver 12 or 12.1. In doing so, the lugs 18.1 and 18.2 engages in to the notches 11.1 and 11.3 in Section 9 of the shaft 2 of the bone screw 1.

In order to prevent the bone screw 1 from falling out of the screw driver 12 or 12.1, it is held through the holder 14 and 14.1 through intervention of the hook 26 in its groove 8. With screw driver 12.1 for accommodating the bone screw 1 the spring element is drawn back in the direction of arrow P, then the bone screw 1 can be used and the spring element is pushed against the direction of arrow P in such a way that the hook 26 engages in to the groove 8 and holds the bone screw 1.

Now, the bone screw 1 can be implanted and/or rotated or screwed-in in a bone and/or in an already cut hole. In the process the bone screw cuts off itself with its threading 6 a thread in the bone tissue.

With the exceedance of a defined torque, the shaft 2 shears at the predetermined breaking point 4 from the screw head 5 and the screw part 3. As a result, an annular shear area is formed, which is not shown here. As can be identifiable from FIG. 3, this lies slightly depressed in the screw head 5. The advantage is that the shear area which is formed mostly as rough area, cannot lead to irritation in the skin or in the flesh.

With the screw driver 12.1, it is especially advantageous that by pushing the holder 14.1 in the opposite direction of the arrow P, the sheared shaft 2 can be ejected by the hook 26 forwards from the screw driver 12.1 and its tubular base part 13.1.

After removing the shaft 2, the screw part 3 with the screw driver 12 or 12.1 is introduced into the pre-cut hole. For this purpose, the screw driver 12 and 12.1 with its lug 19 is introduced in to the notches 11.2 and 11.4 at the screw head 5. The screw part 3 can be removed by changing the direction of rotation out of the bone.

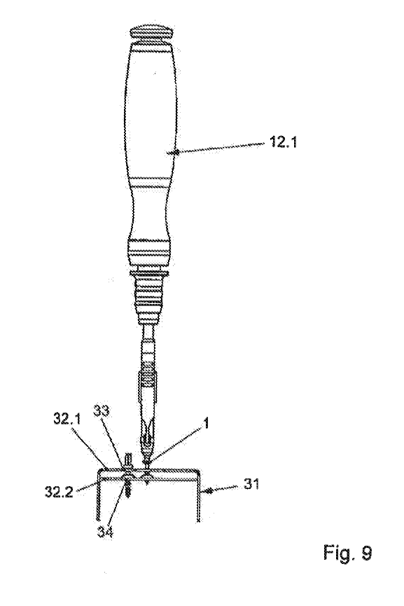

Advantageously, an assembly set as per the invention from the screw driver 12 and/or 12.1 and a bone screw 1 is supplemented through a magazine such as shown in FIG. 9. This magazine 31 serves the purpose, to accommodate the bone screw 1 free of rotation and to prepare for removal with the screw driver 12 and/or 12.1. In order to fulfill this task, it presents two holder plates 32.1 and 32.2. These arranged over each other in a space, which conforms to a height of the screw head 5 and the section 9, and present aligned openings 33 and 34. The opening 33 is created bigger than the largest diameter d₁ of the bone screw 1. The bone screw can be guided through the hole. For this, the opening 34 is shaped in such a way that the bone screw 1 remains hanging with its screw head 5 at the lower holder plate 32.2. Preferably, such a magazine serves the purpose of accommodating many bone screws even in different lengths.

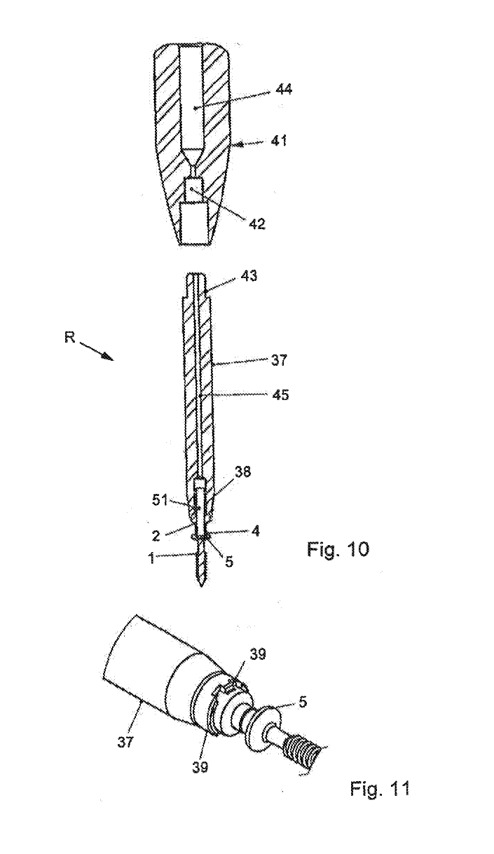

In FIG. 10, another embodiment of a surgical instrument R is shown. This comprises of shaft 2 and bone screw 1. At the bone screw 1, the screw head 5 is formed. Here, the advantage is that the transport and packaging costs can be saved, when the shaft 2 is downsized in shape, so that only the bone screw 1 is delivered with the shaft 2.

In this embodiment, the shaft 2 is introduced for centering in a recess 38 of the rod 37. Besides, the rod 37 possesses notches 43, which co-act with the corresponding engaging lugs 40, as they are recognized in FIG. 13, and bring about rotation of the bone screw 1 through shaft 2.

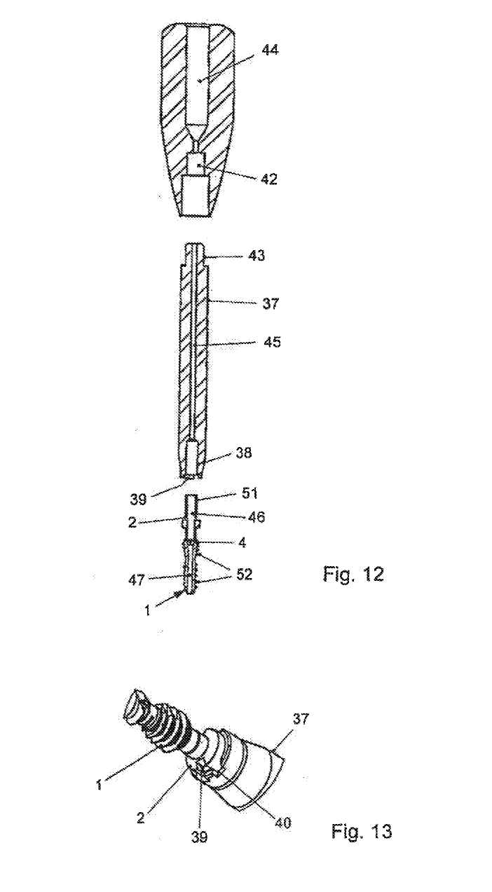

Besides, in FIG. 10 a knob 41 is shown. This knob 41 possesses a recess 42, which serves the purpose of accommodating the rod 37, especially the connector 43. Moreover, how a continuous cannulation is achieved through all the parts shown is recognized in FIG. 10. This continuous cannulation begins with channel 44 in the knob 41, continues in a channel 45 of the rod 37. In order that the cannulation can continue up to the bone screw 1, it is discerned in FIG. 12 well with the help of channel 46 in shaft 2 and a channel 47 in the bone screw.

FIG. 12 shows a bone screw 1, with which the bone screw head 5 is designed without a ridge. This has the advantage that the bone screw 1 can be completely sunk into a bone not shown here. Besides, how the bone screw 1 in FIG. 12 presents multiple grooving 48 is clearly identified.

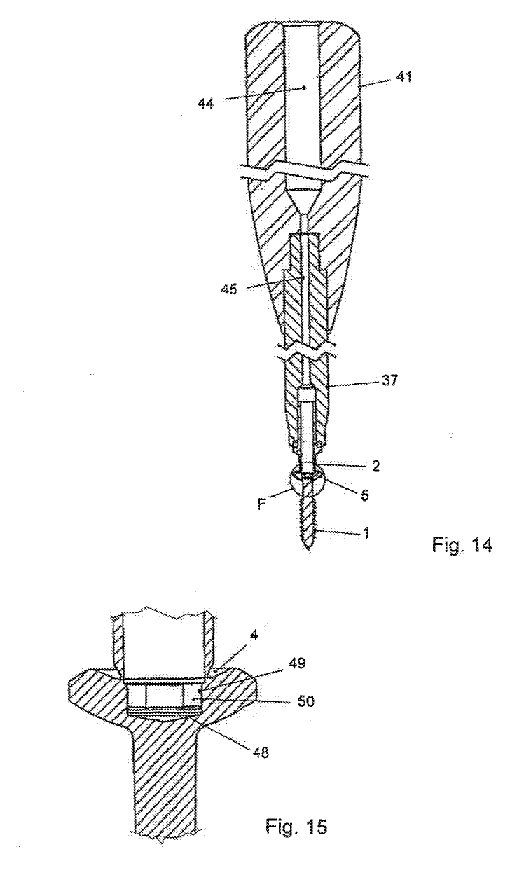

FIG. 14 shows the surgical instrument R, which comprises shaft 2 and bone screw 1, which are connected with each other in the area of the screw head 5 as single part. The area F is again illustrated as enlarged in FIG. 15.

In FIG. 15, how the predetermined breaking point 4 is designed in the form of a depression in annular form is identified. Moreover, an inner serration 50 as well as a driver 49 is shown, which non-positively engage in to each other. In the process the driver is shaped as part of the bone screw 1 and the inner serration 50 as part of the shaft 2. Besides, a recess or additional counter-bore 48 is identified.

1-15. (canceled)

16. A surgical instrument assembly comprising:
a surgical instrument comprising:
a bone screw with a screw head; and
a shaft having a connector portion for screwing the bone screw into a bone;
wherein the shaft with the bone screw is formed integrally and a predetermined breaking point is provided between the shaft and the screw head of the bone screw and the predetermined breaking point is configured break after reaching a specified torque relative the shaft to the screw head; and
a knob or rod configured to removably connect to the connector portion of the shaft or the screw head of the bone screw.

17. The surgical instrument assembly of claim 16, comprising both a knob and a rod, wherein the knob is removably connected to the rod, and the rod is removably connected to the surgical instrument.

18. The surgical instrument assembly of claim 17, wherein the surgical instrument, the knob, and the rod have a plurality of connected channels configured for continuous cannulation of a substance or a filler material from an end of the knob to a tip of the surgical instrument.

19. The surgical instrument assembly of claim 16, wherein the knob or the rod has a spring loaded hook that removably connects to a groove located proximate the end of the shaft of the surgical instrument.

20. The surgical instrument assembly of claim 16, wherein the connector portion of the shaft has a projection with at least two notches that removably connect to at least two lugs located on the rod or knob.

21. The surgical instrument assembly of claim 16, wherein the knob or rod is reusable after attaching the bone screw into the bone via the removable connection between the knob and the shaft of the surgical instrument.

22. The surgical instrument assembly of claim 16, wherein the shaft of the surgical instrument has a bore aligned in a longitudinal direction configured to allow a portion of the knob or the shaft to pass through the bore of the shaft and engage an inner serration located on the screw head.
